(19) United States
(12) Patent Application Publication
(10) Pub. No.: US 2023/0314430 A1
(43) Pub. Date: Oct. 5, 2023

(54) HIGH-THROUGHPUT SEROLOGY ASSAY

(71) Applicant: INANOVATE, INC., Sioux Falls, SD (US)

(72) Inventor: David URE, Wellesley, MA (US)

(21) Appl. No.: 17/996,478

(22) PCT Filed: Apr. 2, 2021
(86) PCT No.: PCT/US2021/025518

§ 371 Date: Oct. 18, 2022

Related U.S. Application Data
(60) Provisional application No. 63/013,988, filed on Apr. 22, 2020.

(51) Int. Cl. G01N 33/569; G01N 33/543

(52) U.S. Cl. CPC G01N 33/56983; G01N 33/54386; G01N 2333/165; G01N 2469/20

(57) ABSTRACT

The invention relates generally to serology assays and, more particularly, to high-throughput serology assays. One aspect of the invention provides a method of detecting a viral antibody in a biological sample of an individual, the method comprising: applying an antigen-containing fluid to an assay surface, the antigen-containing fluid containing an antigen for the vims to be detected and the assay surface containing a biological sample from the individual; removing the antigen-containing fluid from the assay surface; and determining whether the assay surface contains bound antigen.

### CROSS-REFERENCE TO RELATED APPLICATIONS

This application claims priority to co-pending US Provisional Patent Application Ser. No. 63/013,988, filed 22 Apr. 2020, which is hereby incorporated herein as though fully set forth.

### BACKGROUND

Protein microarrays are miniaturized versions of traditional assays enabling high-throughput parallel detection of multiple biomarkers in serum samples or other specimens of interest in a single assay. To survey seroprevalence of virus-specific antibodies, simultaneous detection of antibodies against multiple viruses is also advantageous, and high-throughput serodiagnostic microarray platforms have been developed, or are under active development, for many infectious diseases.

The global spread of the COVID-19 pandemic underlines the importance of worldwide virus surveillance systems. While multiplex PCR assays provide a rapid and specific diagnosis in acute respiratory infections, detection of serum antibodies allows estimating the prevalence of an infection in a population or the determination of immune status and antibody responses in vaccine studies.

For multiplexed immunoassays, fluorescent dyes are frequently used for detection, to help enable high sensitivity of signal detection. These assays have been shown to be suitable for both qualitative and quantitative microarray-based multianalyte assays.

A particularly common serology assay is for the detection of serum IgG antibodies against targeted viruses. For the COVID-19 pandemic, many such serology assays have been developed or are under development. However, the capability to accurately and reliably process very high numbers of patients' samples (many millions in as short a timeframe as possible), as required for an effective pandemic response, is limited.

Indeed, a rapid, low-cost antibody assay with the ability to accurately and reliably screen and classify patients with a virus, or who have had the virus, or who have never had the virus, is a critical need for both present and future pandemic response efforts, and an effectively managed return to economic and societal normalcy.

### SUMMARY

One aspect of the invention provides a method of detecting a viral antibody in a biological sample of an individual, the method comprising: applying an antigen-containing fluid to an assay surface, the antigen-containing fluid containing an antigen for the virus to be detected and the assay surface containing a biological sample from the individual; removing the antigen-containing fluid from the assay surface; and determining whether the assay surface contains bound antigen.

Another aspect of the invention provides a method of detecting the presence or absence of a viral antibody in any of a plurality of biological samples of a plurality of individuals, the method comprising: affixing a first biological sample of a first individual to an assay surface; affixing a second biological sample of a second individual to the assay surface; applying an antigen-containing fluid to the assay surface, the antigen-containing fluid containing an antigen for a virus; removing the antigen-containing fluid from the assay surface; and determining whether the assay surface contains bound antigen.

Still another aspect of the invention provides an assay system comprising: an assay surface; a delivery device for applying an antigen-containing fluid containing an antigen for a virus to the assay surface; and a detection device for detecting the antigen.

Yet another aspect of the invention provides for the use of such an assay system to detect the presence or absence of a viral antibody in a biological sample of at least one individual, including such use in simultaneously or sequentially detecting the presence or absence of the viral antibody in a plurality of biological samples of a plurality of individuals.

Still yet another aspect of the invention provides for the use of such an assay system to detect the presence or absence of a viral antibody in the biological samples of a plurality of individuals, including such use in simultaneously or sequentially detecting the presence or absence of the viral antibody in a plurality of biological samples of the plurality of individuals that are all arrayed on the same test surface or are affixed on a plurality of test surfaces (e.g., on bead surfaces) that are then processed together, either simultaneously or sequentially.

Still another aspect of the invention provides a method of detecting the presence or absence of a viral antibody in any of a plurality of biological samples of a plurality of individuals, the method comprising: affixing a first biological sample of a first individual to a first assay surface; affixing a second biological sample of a second individual to a second assay surface; applying an antigen-containing fluid to the first and second assay surfaces, the antigen-containing fluid containing an antigen for a virus; removing the antigen-containing fluid from the first and second assay surfaces; and determining whether either the first assay surface, the second assay surface, both, or neither contains bound antigen.

### DETAILED DESCRIPTION

Tests that detect and measure antibodies from a patient sample, such as a blood sample, are known as serology tests, and serology testing for COVID-19 is currently in very high demand. The reason for this is the PCR tests currently being used globally to diagnose cases of COVID-19 can only indicate the presence of viral material during infection and will not indicate if a person was infected and has subsequently recovered. Serology tests can be used not only to identify if someone has COVID-19, but also to identify whether people have had the virus in the past, because antibodies released by the patient's immune system remain in the blood long after the virus has left. As such, these tests can better quantify the number of cases of COVID-19, including in those individuals who were asymptomatic or have since recovered.

However, while current microarray technologies facilitate high-throughput immunoassays of antibody detection against multiple pathogens simultaneously, when tasked with detecting a single antibody in many millions of patient samples, the current serology assay approach has significant constraints in time, cost, and sample use.

Typically, a serology assay is presently processed by placing antigens specific to the target virus onto an assay surface (such surface may be an ELISA plate, a microarray, a bead or any other surface used for processing assays). With the antigens on the surface, the patient sample (usually but not always pre-processed into serum or plasma) is then incubated with or otherwise passed across the antigens on the surface. If antibodies (e.g., IgG) to the target virus are present in the patient sample, these should attach to the antigens on the surface. Subsequently, a secondary labelled antibody may be incubated with or passed over the assay surface, and such secondary antibody should in turn attach to the antibody from the patient sample (if present). In this manner, a signal can be detected for any of the secondary labelled antibody that has attached, providing a positive or negative result for the presence or absence of virus antibody in the patient sample. Optionally, the secondary labelled antibody may be replaced either by labelling the patient sample directly, or by using a label free detection method, as will be understood by those skilled in the art.

According to the present invention, instead of placing a virus-specific antigen onto a surface, a patient sample (optionally pre-processed into serum, plasma, total antibody content, IgG content, IgM content, or IgA content) is placed onto a surface. Subsequently, a solution containing antigens to the target virus is incubated or otherwise processed across the patient sample on the surface. In this manner, the antigens should attach to any antibody content related to the virus present in the sample affixed to the surface. The antigen content may be directly labelled (optically, chemically, or otherwise) such that any attachment can be detected with an appropriate analyzer. Optionally, the antigens can be left unlabeled and either a secondary labelled antibody used to build a “sandwich” that can then be detected, or a label-free detection method used to detect attachment of antigen to sample.

This novel approach to serology assays has a critical advantage for pandemic response. Specifically, when applied to a multiplex assay format, wherein multiple patient samples (or the purified IgG content therefrom) can be arrayed (spotted) in parallel, it enables ultra-high patient sample throughput, previously impossible to achieve on a single system.

One embodiment of this is Inanovate's Bio-ID system, which is a novel blood analysis system that enables users to accurately measure the concentration of over 100 blood-based biomarkers in one multiplex test. The Bio-ID was originally designed and built to accurately detect and measure the presence of multiple cancer-related antibodies (tumor autoantibodies) from a patient blood sample. To this end, Inanovate is presently advancing clinical trials for a blood test to diagnose breast cancer using the Bio-ID. The breast cancer test consists of over 50 autoantibody biomarkers, each processed in triplicate. In other words, each breast cancer multiplex test consists of an aggregation of −150 individual tests, each one detecting and measuring the concentration of an autoantibody from a patient blood sample.

This involves placing small quantities of different cancer antigens onto the surface of the Bio-ID test cartridges. This is done through a process called microarray printing, wherein very small ‘spots’ of known proteins are printed onto the surface in known locations (over 150 such spots are printed per test). Subsequently, a patient sample is flowed across the surface of the test cartridge, and if the corresponding antibody biomarker is present in that sample, it attaches to its associated antigen. One is then able to detect this attachment and in turn detect which antibodies are present in that patient sample.

In one embodiment of the invention, instead of printing the antigen onto the assay surface (e.g., the Bio-ID cartridge surface), the antibody (e.g., IgG) content from a patient blood sample is printed. Antibodies can be readily extracted from a blood sample in a simple pre-processing step, as will be apparent to one skilled in the art. Then, a target virus antigen (e.g. the COVID-19 protein (antigen)) is flowed across or otherwise put in contact with the assay surface and any interaction with any of the printed patient antibody samples is detected. This in turn will enable the identification of which of the samples printed onto the assay surface are positive for the target virus (e.g. COVID-19).

By leveraging the high multiplexing capacity of assay systems such as the Bio-ID, one could print many tens of spots of patient sample per test (e.g., using current format 75 patient sample/IgG spots could be printed in duplicate for a total of 150 spots). Each cartridge could run up to 8 tests in −1 hour, and potentially up to 48 or more. This means it would be possible to process on one Bio-ID system one patient sample in one second, at a price point of under $2 per test.

The advantages of such an increase in throughput and decrease in cost compared to existing options are very significant. Any effective near- or medium-term return to normalcy from the COVID-19 pandemic will be at least in part predicated on large scale serology testing. This will provide the data needed to fully understand the penetration and impact of COVID-19 and will provide clarity on how many people have had the virus and are thus likely to be immune to reinfection. A coherent, structured, and effective reopening of the economy can be crafted from such data and knowledge. Indeed, a capability for ultra-high throughput serology testing would provide a powerful foundation for both ongoing and future pandemic responses.

Another embodiment of the invention involves printing or otherwise placing small volumes of the antibody (e.g., IgG) content from multiple different patient blood sample in known and trackable locations on a two-dimensional assay surface such as the surface of a microarray slide or plate. Then, a target virus antigen (e.g. the COVID-19 protein (antigen)) is incubated with or otherwise put in contact with the assay surface and any interaction with any of the printed (or otherwise deposited) patient antibody samples is detected. This in turn will enable the identification of which of the samples printed onto the assay surface are positive for the target virus (e.g., COVID-19). The multiplexing capacity of such an approach could extend into the many hundreds of patient samples (or IgG spots therefrom) per multiplex test.

Yet another embodiment of the invention involves attaching or otherwise placing small volumes of the antibody (e.g., IgG) content from multiple different patient blood sample on different beads (any one bead having a known and trackable patient sample). Then, a target virus antigen (e.g. the COVID-19 protein (antigen)) is incubated with or otherwise put in contact with the beads and any interaction with any of the patient antibody samples is detected. This in turn will enable the identification of which of the samples printed onto the assay surface are positive for the target virus (e.g. COVID-19). Such beads may comprise polystyrene or paramagnetic microspheres as used, for example, in the Luminex® protein assay systems.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

This written description uses examples to disclose the invention, including the best mode, and also to enable any person skilled in the art to practice the invention, including making and using any devices or systems and performing any related or incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A method of detecting a viral antibody in a biological sample of an individual, the method comprising:
applying an antigen-containing fluid to an assay surface, the antigen-containing fluid containing an antigen for the virus to be detected and the assay surface containing a biological sample from the individual;
removing the antigen-containing fluid from the assay surface; and
determining whether the assay surface contains bound antigen.

2. The method of claim 1, wherein applying the antigen-containing fluid includes passing the antigen-containing fluid across the assay surface.

3. The method of claim 1, wherein applying the antigen-containing fluid includes incubating the assay surface in the antigen-containing fluid.

4. The method of claim 1, wherein removing includes passing the antigen-containing fluid across the assay surface.

5. The method of claim 1, wherein removing includes flushing the antigen-containing fluid from the assay surface with an additional fluid.

6. The method of claim 1, wherein the antigen is labeled with at least one of the following: a fluorescent marker, a luminescent marker, a colormetric marker, or a radioactive marker.

7. (canceled)

8. The method of claim 1, wherein detecting includes label-free detecting.

9. The method of claim 1, wherein the biological sample from the individual is selected from a group consisting of: saliva, whole blood, blood serum, blood plasma, total blood antibodies, immunoglobulin G (IgG) isolated from blood, immunoglobulin M (IgM) isolated from blood, and immunoglobulin A (IgA) isolated from blood.

10. The method of claim 1, further comprising, after the removing step and before the determining step:
applying a labeling fluid to the assay surface, the labeling fluid containing a labeled secondary antibody capable of binding to the antigen; and
removing the labeling fluid from the assay surface.

11-15. (canceled)

16. The method of claim 1, wherein the assay surface includes a plurality of biological samples from a plurality of individuals.

17. The method of claim 16, wherein determining includes simultaneously determining whether the antigen is bound to each of the plurality of biological samples.

18. The method of claim 16, wherein determining includes sequentially determining whether the antigen is bound to each of the plurality of biological samples.

19-22. (canceled)

23. A method of detecting the presence or absence of a viral antibody in any of a plurality of biological samples of a plurality of individuals, the method comprising:
affixing a first biological sample of a first individual to an assay surface;
affixing a second biological sample of a second individual to the assay surface;
applying an antigen-containing fluid to the assay surface, the antigen-containing fluid containing an antigen for a virus;
removing the antigen-containing fluid from the assay surface; and
determining whether the assay surface contains bound antigen.

24-31. (canceled)

32. An assay system comprising:
at least one assay surface;
a delivery device for applying an antigen-containing fluid containing an antigen for a virus to the at least one assay surface; and
a detection device for detecting the antigen.

33. (canceled)

34. (canceled)

35. The assay system of claim 32, wherein the at least one assay surface includes a two-dimensional array suitable for carrying a plurality of arrayed biological samples.

36. The assay system of claim 35, wherein the two-dimensional array is suitable for carrying at least one hundred biological samples.

37. The assay system of claim 35, wherein the two-dimensional array is suitable for carrying at least fifty biological samples.

38. The assay system of claim 35, wherein the two-dimensional array is suitable for carrying at least ten biological samples.
